(19) United States
(12) Patent Application Publication
(10) Pub. No.: US 2020/0238071 A1
(43) Pub. Date: Jul. 30, 2020

(54) CONNECTORS FOR ALLOWING AN ENGAGEMENT AND FLUID PASSAGEWAY BETWEEN MEDICAL VESSELS

(71) Applicant: Mikael NAYGAUZ, Forrest Hills, NY (US)

(72) Inventor: Mikael NAYGAUZ, Forrest Hills, NY (US)

(21) Appl. No.: 16/278,053

(22) Filed: Feb. 16, 2019

Related U.S. Application Data
(60) Provisional application No. 62/797,361, filed on Jan. 28, 2019.

(51) Int. Cl. A61M 39/16; A61J 1/20; A61M 5/178; A61J 1/10; A61M 5/14; A61M 39/10

(52) U.S. Cl. CPC A61M 39/165; A61J 1/2055; A61J 1/2089; A61M 39/1011; A61M 5/1782; A61J 1/10; A61M 5/14; A61J 1/2096

(57) ABSTRACT

The present invention relates, in some embodiments thereof, to connectors having cavities and systems comprising same for allowing an engagement and fluid passageway between medical vessels. In some embodiments, the connectors allow repeatable fluid transfer between vessels. In some embodiments, the connectors configured to connect between a plurality of medical vessels.

### CROSS REFERENCE TO RELATED APPLICATIONS

The present application claims priority to U.S. Provisional Application No. 62/797,361 filed on Jan. 28, 2019. The entire contents of this application is expressly incorporated herein by reference.

### FIELD OF THE INVENTION

The present invention relates, in some embodiments thereof, to connectors and systems comprising same for allowing an engagement and fluid passageway between medical vessels. In some embodiments, the connectors allow repeatable fluid transfer between vessels. In some embodiments, the connectors configured to connect between a plurality of medical vessels.

### BACKGROUND OF THE INVENTION

The medicinal practice routinely involves administration of medical substances, such as, medicaments, fluids, nutritional substances and the alike, to patients or animals. The preparation and/or administration of such medicinal substances typically involves one or more transfers of those substances between pharmaceutical vessels or administration devices (such as, vials, syringes, infusion lines, connectors, etc.). Each such act of transferring substances between vessels or devices exposes the connection interfaces of the vessels and accordingly the medical substances themselves to contaminants present in ambient air or ambient air particles (e.g., bacteria, viruses, funguses, spores, pyrogens, dirt). In addition, connection interfaces are further prone to contaminations due to physical contact of the interfaces, for example, with nonsterile gloves, or devices.

Such contaminations are a major problem in the healthcare setting since contaminants, once invading within medicinal substances, may pose substantial danger if administered intracorporeally to patients.

Typical connection interfaces of pharmaceutical vessels or pharmaceutical administration devices include rubber bungs and/or stoppers covered by a cap and/or seal that can be flicked off and/or are removed prior to usage thereof. These rubber bungs/stoppers are used to allow penetration by a needle attached to a syringe or by other medical connectors. When the cap and/or seal is flicked off and/or removed, the rubber bung and/or stopper is exposed to ambient air and to contaminants present therein. Accordingly, exposure of connection interfaces to ambient air may involve contamination of the interfaces and consequently contamination of a beneficial substance to be provided to a patient.

Existing systems include USD720,067; USD717,947; USD703,812; USD690,418; USD639,939; USD637,713; U.S. Pat. Nos. 9,790,011; 9,775,777; 9,561,326; 9,493,281; 9,492,353; 9,309,020; 9,173,816; 9,168,203; 9,162,803; 9,039,672; 8,926,583; 8,827,978; 8,790,330; 8,662,985; 8,657,803; 8,622,985; 8,562,583; 8,545,475; 8,523,838; 8,491,563; 8,480,646; 8,449,521; 8,381,776; 8,336,587; 8,328,772; 8,287,513; 8,225,826; 8,075,550; 8,029,747; 7,998,134; 7,975,733; 7,942,860; 7,867,215; 7,744,581; 7,731,678; 7,387,216; 7,306,584; 6,875,203; 6,729,370; 6,715,520; 6,602,239; 6,409,708; 6,343,629; 6,162,199; 6,113,583; 6,063,068; 5,893,397; 5,876,380; 5,832,971; 5,807,374; 5,746,733; 5,569,235; 5,462,535; 5,405,326; 5,292,318; 5,279,582; 4,944,723; 4,932,947; 4,932,937; 4,919,657; 4,915,701; 4,826,489; 4,673,404; 4,564,054; 3,610,241; 3,605,743; 3,587,575; 3,583,399; 3,578,037; 3,556,099; 3,552,387; 3,406,686; 3,380,450; 3,375,825; 3,342,180; 3,330,282; 3,330,281; 3,306,290; 3,255,752; 3,253,592; 3,076,456; 2,972,991; 2,922,419; US20160262982; US20160038373; US20150209568; US20140183196; US20140016570; US20140007973; US20140000754; US20130184672; US20130006200; US20120209238; US20120209218; US20120203194; US20110284561; US20110186177; US20110125128; US20110108158; US20110098647; US20100249745; US20100198182; US20100152669; US20100147402; US20100036319; US20100004602; US20090057258; US20080312634; US20080223484; US20080171981; US20060276759; US20050215976; US20030199847; US20030187420; US20020130100; US20020115981; US20020099354; ES2577377T3; EP2852367B1; EP2666513; EP2155141B1.

In order to overcome this obstacle, the current medical practice involves swabbing the surface of a connection interface with a disinfecting agent, such as 70% isopropyl alcohol, prior to accessing the connection interface. Other methods include i.v. (intravenous) rooms which are used for the sterile preparation of i.v. medications. Such rooms, to keep medicinal preparations as sterile as possible, are equipped with special instruments including, hoods with air filtration systems (e.g., HEPA filters), ventilation systems and air pressure systems. Additionally, those rooms necessitate that the medical staff working in these rooms are properly garmented, are properly trained, and require aseptic techniques, and employ quality control and validation processes. These systems require regular upkeep by certified personnel and require regular cleaning. These systems are therefore expensive, labor intensive, and require regular maintenance and testing to assure that they are operating effectively. The above described systems and methods are either cumbersome and expensive or inefficient in addressing the problem of reducing/eliminating contaminants on connection interfaces.

Taken together, the above described systems and methods are either cumbersome and expensive or inefficient in addressing the problem of reducing/eliminating contaminants on connection interfaces.

Furthermore, typical connectors used to connect intravenous (IV) bags to vials, containers to containers, and/or containers to medical devices comprise a ratchet teeth mechanism or other retention mechanism which grasps a port of a bag, a port of a vial, a port of a container, and/or a port of a medical device. This results in a connection between the bag and the vial (via the connector), a container and a container (via a connector), and/or a container and medical device (via a connector) that is sufficient to hold the vial to the bag. However, there have been instances where the bag and the vial may disconnect either spontaneously, if the bag and vial fall to the floor, and disconnection may occur by a force of a user's hand. As a result, a connector that only grasps a vial “port”, a bag “port”, a container “port”, and/or a medical device “port” does not provide a connection that is very secure. This is of particular concern when toxic substances such as chemotherapeutic medicaments are housed in the vials because disconnection of a vial containing a toxic/chemotherapeutic substance from a bag/container/device may result in the leakage of the toxic/chemotherapeutic substance into the environment exposing the surroundings and a user to the toxic substance. Additionally, currently used connectors used for connecting two vessels, two medical devices, two containers, and/or a vessel to a medical device do not protect the vessel or the device. Therefore, if the vessel and/or container and/or device falls to the ground when connected to the connector the vessel/device/container/vial may break and release the toxic/chemotherapeutic substance from the vessel/device/container/vial thus contaminating the environment with a toxic dangerous substance and exposing a user to the toxic substance. Therefore, there is a dire need in the current state of the art to provide better connectors that will guarantee a connection between at least two vessels that will prevent disconnection of two vessels from each other spontaneously, upon accidental falling to the ground, and/or via manipulation/force of a user's hand. Additionally, there is a need in the art for connectors that will prevent breakage of a vessel and/or device upon falling to the ground.

There is furthermore a need for reliable, user friendly and cost-effective solutions allowing a secure fluid transfer and engagement between vessels. The herein described invention provides a solution to such long felt needs.

### SUMMARY OF THE INVENTION

It is an object of the invention to provide connectors, systems and methods which allow vessels to fluidly communicate with each other.

It is an object of the invention to provide connectors, systems and methods which allow to transfer medical substances between vessels in a secure manner.

It is an object of the invention to provide connectors, systems and methods which allow to repeatedly transfer medical substances between vessels.

It is an object of the invention to provide connectors, systems and methods which allow to securely load/reload a vessel with a medical substance.

It is an object of one or more embodiments of the invention to overcome limitations in the prior art in relation to connectors typically used by healthcare personnel to connect pharmaceutical vessels, containers, and medical devices to each other.

It is an object, of one or more embodiments of the invention, to provide a connector having at least one cavity configured to accommodate at least a portion of a body of a container.

It is an object, of one or more embodiments of the invention, to provide a connector having at least one cavity that will protect a container and prevent breakage/cracking of the container during the preparation, delivery, and/or administration of a beneficial substance if the connector-container falls to the ground.

Objects of the invention are achieved by providing connectors, methods and systems comprising same which allow an engagement between vessels that are otherwise un-engageable. Objects of the invention are achieved by providing connectors and systems comprising same which allow vessels to connect and fluidly communicate.

The herein disclosed connectors demonstrate simplicity, reliability and provide a safe engagement between two to more vessels, and/or cartridges.

In a general aspect of the embodiments, the invention provides a connector having at least one cavity, the connector configured to connect two vessels to each other, the at least one cavity capable of accommodating at least a portion of a container's body.

In a general aspect of the embodiments, the invention provides connector(s) having at least one cavity, the cavity characterized in that it is capable of accommodating at least a portion of a container's body.

In one or more embodiments, the connector is configured to connect two containers to each other. In one or more embodiments, the connector is configured to couple two containers to each other. In one or more embodiments, the connector is configured to engage two containers to each other. In one or more embodiments, the connector is configured to engage a container to a medical device.

In one or more embodiments, the connector is configured to have at least one cavity that will accommodate at least 35% the length of a container. In one or more embodiments, the connector is configured to have at least one cavity that will accommodate at least 50% the length of a container. In one or more embodiments, the connector is configured to have at least one cavity that will accommodate at least 75% the length of a container. In one or more embodiments, the connector is configured to have at least one cavity that will accommodate at least 90% the length of a container. In one or more embodiments, the connector is configured to have at least one cavity that will accommodate at least 100% the length of a container. In one or more embodiments, the connector is configured to have at least one cavity that will accommodate an entire length of a container.

In one or more embodiments, the connector is configured to have at least one cavity that will accommodate at least 35% the width of a container. In one or more embodiments, the connector is configured to have at least one cavity that will accommodate at least 50% the width of a container. In one or more embodiments, the connector is configured to have at least one cavity that will accommodate at least 75% the width of a container. In one or more embodiments, the connector is configured to have at least one cavity that will accommodate at least 90% the width of a container. In one or more embodiments, the connector is configured to have at least one cavity that will accommodate at least 100% the width of a container. In one or more embodiments, the connector is configured to have at least one cavity that will accommodate an entire width of a container.

In one or more embodiments, the connector is configured to have at least one cavity that will accommodate at least 35% the volume of a container. In one or more embodiments, the connector is configured to have at least one cavity that will accommodate at least 50% the volume of a container. In one or more embodiments, the connector is configured to have at least one cavity that will accommodate at least 75% the volume of a container. In one or more embodiments, the connector is configured to have at least one cavity that will accommodate at least 90% the volume of a container. In one or more embodiments, the connector is configured to have at least one cavity that will accommodate at least 100% the volume of a container. In one or more embodiments, the connector is configured to have at least one cavity that will accommodate an entire volume of a container.

In one or more embodiments, the container is a bag. In one or more embodiments, the container is a bottle. In one or more embodiments, the container is a syringe.

In one or more embodiments, the container is a cartridge having at least one protective side wall. In one or more embodiments, the container has a circumferential protective side wall. In one or more embodiments, the container has a plunger. In one or more embodiments, the container has a plunger and at least one protective side wall. In one or more embodiments, the container has a moveable wall. In one or more embodiments, the container has an expulsion member. In one or more embodiments, the expulsion member is a plunger. In one or more embodiments, the container has a plunger.

In one or more embodiments, the connector further configured to enter a cavity of a bag. In one or more embodiments, the connector further configured to enter a cavity of a bottle. In one or more embodiments, the connector further configured to enter a cavity of a container.

In one or more embodiments, the connector further comprising a locking mechanism configured to lock a container to the connector. In one or more embodiments, the locking mechanism comprising a spring mechanism. In one or more embodiments, the locking mechanism comprising a spring mechanism and a retractable/extended retention member. In one or more embodiments, the locking member comprising a retractable/extendable retention member. In one or more embodiments, the locking mechanism disposed at about the opening of the connector's cavity. In one or more embodiments, the locking mechanism configured to secure the bottom of the container to the connector.

In one or more embodiments, the spring and retractable/extended retention member configured to hold the bottom of the container. In one or more embodiments, the locking mechanism of the connector being a ratchet teeth mechanism. In one or more embodiments, the ratchet teeth mechanism disposed on the interior side wall of the cavity of the connector. In one or more embodiments, the ratchet teeth mechanism disposed at about the opening of the cavity of the connector. In one or more embodiments, the ratchet teeth mechanism disposed at the opening of the cavity of the connector. In one or more embodiments, the ratchet teeth mechanism configured to grasp the bottom of the container when the container is engaged to the connector.

In one or more embodiments, the connector further comprising an engagement mechanism in the form of a dent, the dent configured to engage/couple/accommodate a wing of the container. In one or more embodiments, the wing is retractable/extendable allowing the container to enter the cavity of the connector. In one or more embodiments, the wing of the container is operable in at least two positions, a retractable position that allows the container to enter the connector and an extended position that locks the container inside the connector's cavity.

In one or more embodiments, the connector is further configured to enter a cavity of a bag/bottle while the connector houses/contains a container. In one or more embodiments, the connector is further configured to enter a cavity of a container. In one or more embodiments, the connector is further configured to enter a cavity of a bag. In one or more embodiments, the connector is further configured to enter a cavity of a bottle.

In one or more embodiments, the connectors allow an engagement between a cartridge and a vessel. In one or more embodiments, the connectors allow an engagement between a cartridge and another cartridge. In one or more embodiments, the connectors allow an engagement between a vessel and another vessel. In one or more embodiments, a plurality of cartridges can be connected to a vessel via the herein disclosed connectors. In one or more embodiments, a plurality of cartridges can be connected via the herein disclosed connectors. In one or more embodiments, a plurality of vessels can be connected via the herein disclosed connectors.

Thus, the present invention provides a system for the engagement and fluid passageway between vessels, the system comprising: a first vessel; a second vessel; and a connector having at least one cavity, the cavity configured to allow entry of at least one of the first vessel's body and the second vessel's body, the connector configured to allow a connection between the first vessel and the second vessel; wherein the first and second vessels connected to each other via the connector and wherein each of the first vessel, second vessel and connector includes an aperture and/or a seal for allowing the fluid passageway between the first vessel and the second vessel.

In one or more embodiments, the connector includes at least one cavity, the cavity configured to accommodate therein the first vessel, second vessel and/or a portion thereof.

In one or more embodiments, the connector includes a first cavity for accommodating therein the first vessel and a second cavity for accommodating therein the second vessel, thereby allowing the vessels' connection and fluid passageway via the connector.

In one or more embodiments, the first and/or second vessels include a cavity for accommodating therein the connector or a portion thereof, thereby allowing the vessels connection and fluid passageway via the connector.

In one or more embodiments, the connector includes a cavity for accommodating therein the first vessel and wherein the second vessel includes a cavity for accommodating therein the connector, thereby allowing the vessels' connection and fluid passageway via the connector.

In one or more embodiments, the system comprises a plurality of first vessels and a plurality of connectors, such that each first vessel configured to connect a connector and the second vessel configured to connect the plurality of connectors and vessels. In one or more embodiments, the second vessel includes at least one cavity for accommodating therein at least one of the connectors. In one or more embodiments, at least one of the connectors includes a cavity for accommodating therein at least one of the first vessels.

In one or more embodiments, the vessel is selected from the group consisting of a bottle, a syringe, an infusion line, a connector, a filter, a manifold, a bag port, a bottle port, a vial port, a cartridge and a combination thereof. In one or more embodiments, the first vessel is a cartridge configured to fill the second vessel with a fluidic or semi fluidic substance.

In one or more embodiments, when connected, a wall of at least one of the first vessel and second vessel extends from the boundaries of a wall of the connector. In one or more embodiments, when connected, a wall of at least one of the first vessel and second vessel is aligned with the boundaries of a wall of the connector. In one or more embodiments, when connected, a wall of at least one of the first vessel and second vessel is concealed within the walls of the connector.

In one or more embodiments, when connected, a wall of the connector and/or the first vessel extends from the boundaries of a wall of the second vessel. In one or more embodiments, when connected, a wall the connector and/or the first vessel is aligned with the boundaries of a wall of the second vessel. In one or more embodiments, when connected, a wall the connector and/or the first vessel is concealed within the walls of the second vessel.

In one or more embodiments, the first vessel and/or second vessel includes a fluid transfer mechanism for transferring a fluidic or semi fluidic substance from the first vessel to the second vessel or vice versa. In one or more embodiments, the fluid transfer mechanism includes a moveable wall which when pushed towards an aperture of the vessel, forcibly pushes the fluidic or semi fluidic substance from the vessel. In one or more embodiments, the fluidic or semi fluidic substance is a liquid. In one or more embodiments, the fluidic or semi fluidic substance is a powdered solid. In one or more embodiments, the fluidic or semi fluidic substance is a lyophilized powder. In one or more embodiments, the fluidic or semi fluidic substance is a gas.

In one or more embodiments, the system further comprises a locking mechanism configured to secure the engagement between the vessel and connector. In one or more embodiments, the locking mechanism comprises a securing member selected from a hook, a tooth, a pin, a ratchet tooth, a thread, a ball, a bangle, a locking key, a knob, a luer, a retention member, an adhesive, a clamping member, a zip-tie and a combination thereof. In one or more embodiments, the securing member is locked into a locking member selected from a dent, a hole, a channel, and a slot. In one or more embodiments, the locking member is retractable and moveable between a retracted position wherein the locking member abuts or inserted within a wall of the vessel or connector and an extended position, wherein the locking member protrudes from a wall of the vessel or connector. In one or more embodiments, the locking member is spring loaded or attached to a spring, allowing planar movement of the locking member.

In another aspect, the present invention provides a connector for the engagement and establishment of a fluid passageway between vessels, the connector configured to connect a first vessel and a second vessel and includes at least one cavity for accommodating therein at least one of the first and second vessels, thereby allowing an engagement and a fluid passageway between the first and second vessels.

In another aspect, the present invention provides a connector having a cavity, the cavity characterized in that is allows entrance of at least a portion of a container into the cavity.

In one or more embodiments, the cavity accommodates at least 35% of at least one of a container's length and width.

In one or more embodiments, the cavity accommodates at least 50% of at least one of a container's length and width.

In one or more embodiments, the cavity accommodates at least 75% of at least one of a container's length and width.

In one or more embodiments, the cavity accommodates at least one of an entire container's length and width.

In one or more embodiments, the container is a vial.

In one or more embodiments, the container is a bottle.

In one or more embodiments, the container is a bottle with a plunger.

In one or more embodiments, the container is a cartridge with a protective side wall.

In another aspect, the present invention provides a connector having a cavity, the cavity configured to engulf at least a portion of a container's length.

In one or more embodiments, the cavity engulfs at least 25% of a container's length.

In one or more embodiments, the cavity engulfs at least 50% of a container's length.

In one or more embodiments, the cavity engulfs at least 75% of a container's length.

In one or more embodiments, the cavity engulfs the entire length of a container.

In one or more embodiments, the connector further comprising a piercing member.

In one or more embodiments, the piercing member is a needle.

In one or more embodiments, the piercing member is a hollowed needle.

In one or more embodiments, the connector further comprising a frangible seal.

In one or more embodiments, the present invention provides a method of connecting and establishing a fluid passageway between vessels, the method comprising the steps of: connecting a first vessel to a connector, the connector having a cavity, the cavity characterized in that is allows at least a portion of the first vessel's body to enter the cavity; connecting a second vessel to the connector; and allowing a fluid passageway between the first vessel and the second vessel via the connector.

In one or more embodiments, an airtight seal is formed between the vessels and the connector following the step of connection, allowing a hermetically sealed connection between said first vessel and said second vessel.

In one or more embodiments, the method further comprising locking and/or engaging the first and/or second vessel to the connector via a locking mechanism. In one or more embodiments, the step of locking the vessel to a connector includes coupling a locking member to a securing member. In one or more embodiments, locking of said first vessel to said second vessel is via a push, a twist, a slide activation, or combinations thereof.

In one or more embodiments, the method further comprising transferring a fluid substance between the vessels after connecting the vessels.

In one or more embodiments, the step of connecting a vessel includes inserting the vessel into a cavity within the connector. In one or more embodiments, the method comprises connecting a first vessel into a first cavity within the connector and further connecting a second vessel into a second cavity within the connector.

In one or more embodiments, the step of connecting a vessel includes inserting the connector into a cavity within the vessel. In one or more embodiments, the step of connecting the vessel includes inserting the connector into the cavity within the second vessel. In one or more embodiments, the step of connecting a vessel includes inserting a connector into a cavity within a second vessel.

In one or more embodiments, the method comprises connecting a first vessel into a cavity within the connector and further connecting the connector into a cavity within the second vessel. In one or more embodiments, the method comprises connecting a plurality of connectors to a plurality of first and/or second vessels. In one or more embodiments, the method comprises a second vessel having a plurality of cavities disposed within the second vessel, the plurality of the cavities configured to allow entry of a plurality of connectors into the cavities.

Unless otherwise defined, all technical or/and scientific terms used herein have the same meaning as commonly understood by one of ordinary skill in the art to which the invention pertains. Although methods and materials similar or equivalent to those described herein can be used in the practice or testing of embodiments of the invention, exemplary methods or/and materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1B are front cut view illustrations demonstrating a system with a first vessel, a second vessel and a connector for connecting and allowing a fluid communication between the vessels, the vessels have side walls which outwardly extend from the side walls of the connector, according to some embodiments of the invention.

FIGS. 2A-2B are front cut view illustrations demonstrating a system with a first vessel, a second vessel and a connector for connecting and allowing a fluid communication between the vessels, the vessels have side walls which are aligned with the side walls of the connector, according to some embodiments of the invention.

FIGS. 3A-3B are front cut view illustrations demonstrating a system with a first vessel, a second vessel and a connector for connecting and allowing a fluid communication between the vessels, the vessels have side walls which are shorter from and concealed within the side walls of the connector, according to some embodiments of the invention.

FIGS. 4A-4B are front cut view illustrations demonstrating a system which further includes a locking mechanism with a retractable stopper for locking the vessels within the connector, according to some embodiments of the invention.

FIGS. 5A-5B are front cut view illustrations demonstrating a system with a first vessel in the form of a bag, a second vessel in the form of a cartridge and a connector, the connector includes a first cavity for accommodating the body of the cartridge and another cavity for accommodating a port of the bag, according to some embodiments of the invention.

FIGS. 6A-6B are front cut view illustrations demonstrating a system with a first vessel in the form of a bag, a second vessel in the form of a cartridge and a connector, the system includes a locking mechanism in the form of side tabs, according to some embodiments of the invention.

FIGS. 7A-7C are bottom view illustrations of a connector with a locking mechanism (FIG. 7A), a vessel that engages the connector (FIG. 7B), and a vessel when introduced within the connector and locked by the locking mechanism (FIG. 7C), according to some embodiments of the invention.

FIG. 8 is a side cut view illustration of a system with a plurality of connectors and first vessels, for connecting to a second vessel, the connectors and first vessels include side walls which are aligned with side walls of the second vessel, according to some embodiments of the invention.

FIG. 9 is a side cut view illustration of a system with a plurality of connectors and first vessels, for connecting to a second vessel, the connectors and first vessels include a moveable wall to provide for the fluid transfer between vessels, according to some embodiments of the invention;

FIG. 10 is a side cut view illustration of a system with a plurality of connectors and first vessels, for connecting to a second vessel, the connectors and first vessels include side walls which extend from the side walls of the second vessel, according to some embodiments of the invention.

FIG. 11 is a side cut view illustration of a system with a plurality of connectors and first vessels, for connecting to a second vessel, the system includes a locking mechanism in the form of threads and complementary threads and a locking mechanism in the form of tooth and dents, according to some embodiments of the invention.

FIG. 12 is a side cut view illustration of a system with a plurality of connectors and first vessels, for connecting to a second vessel, the system includes a locking mechanism in the form of retractable stoppers, according to some embodiments of the invention.

FIG. 13 is a side cut view illustration of a system with a plurality of connectors and first vessels, for connecting to a second vessel, the system includes an engagement mechanism in the form of retractable stoppers, and teeth locked into dents, according to some embodiments of the invention.

FIG. 14 is a side cut view illustration of a system with a plurality of connectors and first vessels having a form of bottles, for connecting to a second bag-like vessel, the connectors configured to accommodate or engulf the bottles, the side walls of the vials are aligned with the side walls of the connector and second vessel, according to some embodiments of the invention.

FIG. 15 is a side cut view illustration of a system with a plurality of connectors and first vessels having a form of bottles, for connecting to a second bag-like vessel, the connectors configured to accommodate or engulf the bottles, the side walls of the bottles are concealed within the side walls of the second vessel, according to some embodiments of the invention.

FIG. 16 is a side cut view illustration of a system with a second vessel in the form of a bag, and a plurality of connectors with a plurality of first vessels having a form of syringes, the connectors configured to accommodate or engulf the syringes, the side walls of the syringes are aligned with the side walls of the second vessel, according to some embodiments of the invention.

FIG. 17 is a side cut view illustration of a system with a second vessel in the form of a bag, and a plurality of connectors with a plurality of first vessels having a form of syringes, the connectors configured to accommodate or engulf the syringes, the side walls of the syringes are entirely inside and concealed within the side walls of the second vessel, according to some embodiments of the invention.

It should be appreciated that for simplicity and clarity of illustration, elements shown in the figures have not necessarily been drawn to scale. For example, the dimensions of some of the elements are exaggerated relative to each other for clarity. Further, where considered appropriate, reference numerals have been repeated among the figures to indicate corresponding elements.

### DETAILED DESCRIPTION OF THE INVENTION

It is understood that the invention is not limited to the particular methodology, devices, items or products etc., described herein, as these may vary as the skilled artisan will recognize. It is also to be understood that the terminology used herein is used for the purpose of describing particular embodiments only and is not intended to limit the scope of the invention. The following exemplary embodiments may be described in the context of exemplary connector for ease of description and understanding. However, the invention is not limited to the specifically described products and methods and may be adapted to various applications without departing from the overall scope of the invention. All ranges disclosed herein include the endpoints. The use of the term “or” shall be construed to mean “and/or” unless the specific context indicates otherwise.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

It is understood that the invention is not limited to the particular methodology, devices, items or products etc., described herein, as these may vary as the skilled artisan will recognize. It is also to be understood that the terminology used herein is used for the purpose of describing particular embodiments only and is not intended to limit the scope of the invention. The following exemplary embodiments may be described in the context of exemplary medical devices/containers/systems for ease of description and understanding. However, the invention is not limited to the specifically described products and methods and may be adapted to various applications without departing from the overall scope of the invention. All ranges disclosed herein include the endpoints. The use of the term “or” shall be construed to mean “and/or” unless the specific context indicates otherwise.

The embodiments are described more fully hereinafter with reference to the accompanying drawings, in which embodiments of the inventive concept are shown. In the drawings, the size and relative sizes of layers and regions can be exaggerated for clarity. Like numbers refer to like elements throughout. The embodiments can, however, be embodied in many different forms and should not be construed as limited to the embodiments set forth herein. Rather, these embodiments are provided so that this disclosure will be thorough and complete, and will fully convey the scope of the inventive concept to those skilled in the art. The following embodiments are discussed, for simplicity, with regard to the terminology and structure of a medical services environments, such as a hospital, nursing home, doctor's office, walk-in clinic, or home infusion setting.

Reference throughout the specification to “one embodiment” or “an embodiment” means that a particular feature, structure, or characteristic described in connection with an embodiment is included in at least one embodiment of the embodiments. Thus, the appearance of the phrases “in one embodiment” or “in an embodiment” in various places throughout the specification is not necessarily referring to the same embodiment. Further, the particular feature, structures, or characteristics can be combined in any suitable manner in one or more embodiments.

The present invention pertains to connectors, methods and systems for allowing connection between one or more vessels. In one or more embodiments, the connectors allow a fluid passageway between the connected vessels. In one or more embodiments, the connectors allow a connection and fluid passageway between vessels which are otherwise un-engageable.

Optionally, the connectors allow a repeated use thereof. Optionally, the connectors allow to refill vessels with cartridges. Optionally, the connectors are connected to the vessels via cavities within the connectors. In accordance with this embodiment, the cavities are sized and shaped to accommodate the body of the vessel. In one or more embodiments, the cavities are sized and shaped to accommodate at least a portion of the body of the vessel. Additionally, or alternatively, the vessel includes a cavity for accommodating therein the connector. In one or more embodiments, the vessel includes a cavity for accommodating at least a portion of the connector. In one or more embodiments, the vessel includes a cavity for accommodating an entire length and/or width of the connector.

As used herein the term “vessel” refers to any device utilized for containing, transferring and/or administering substances. In one or more embodiments, the vessels may be used for containing medical substances. In an embodiment of the invention, the vessel is a medical vessel. In one or more embodiments, the term “medical vessel” is interchangeable with the term “pharmaceutical vessel”. In an embodiment of the invention, the vessel is a medical device. In an embodiment of the invention, the vessels are used for, and adapted to allow connection to another vessel or to a connector. In an embodiment of the invention, the vessel is utilized for containing or housing a medical substance. In one or more embodiments, the medical substance may be a medicament. In one or more embodiments, the medicament may be an injectable medicament. In one or more embodiments, the medicament may be a sterile medicament, nutritional substance, a diagnostic substance, a diluent, a sterile fluid, and combinations thereof. Various types of vessels are contemplated. The vessel may be selected, without limitation, from a vial, a bag, a chamber, a bottle, a cartridge and the alike. In an embodiment of the invention, the term vessel further encompasses elements that can be used to connect between vessels. In accordance with this embodiment, the vessel may be selected, without limitation, from a connector, a connector having a plurality of openings, a connector having a cavity, a syringe, an infusion line, a tubing, an infusion line, a spike, a syringe, a filter, a port and a manifold.

In one or more embodiments, the connector is applicable for connecting two or more vessels. In one or more embodiments, the connector is applicable for allowing connection between two or more vessels which are otherwise un-engageable. In one or more embodiments, the connector is applicable for refilling one or more vessels with a fluid or semi-fluid substance. In one or more embodiments, the vessel is a cartridge.

As used herein the term “fluid communication” refers to two or more vessels (e.g., a cartridge) in which substances may pass therethrough either directly or indirectly. The fluid communication may occur via a fluid passageway that allows for the flow/transfer of substances. In an embodiment of the invention, fluid passageway between vessels is established when any seals or covers of the apertures of the herein disclosed systems and/or of the vessels and/or of a connector are open. Optionally, the vessels and/or connectors of the systems are provided with seals/covers and those seals/cover become open upon piercing by a piercing member or breaking/rupturing thereof. Optionally, the vessels and/or apertures of the systems are provided with seals/covers and those seals/covers are opened upon uncovering or removing thereof by a user. Optionally, one or more of the seals/covers disclosed herein are fragile, allowing piercing thereof with a sharp instrument (i.e., a piercing member) or by the application of a sufficient force to break or rupture the seals/covers. The piercing member may optionally be a needle, for example, a hollowed needle, but other sharp elements are contemplated. In one or more embodiments, the seals/covers may be peelable. In one or more embodiments, the seals/covers may be frangible.

In an embodiment of the invention, the herein disclosed connectors, systems and methods allow fluid communication between two or more, three or more, four or more, five or more, six or more, or seven or more vessels. In an embodiment of the invention, the herein disclosed connectors, systems and methods allow fluid communication in a contaminant-free, or in a substantially contaminant-free manner. For example, between two, three or more, four or more, five or more or six or more vessels. For example, the connectors, systems, and methods of the invention allow fluid communication between two cartridges, two bags, between two containers, between two bottles, between a syringe and a bag, between a syringe and a container, between a syringe and a vial, between a connector and a bag, between a container and a bag, between a vial and a bag, between a container and an infusion line or between a syringe and a connector. In an embodiment of the invention, the herein disclosed connectors and systems are disposable. In an embodiment of the invention, the herein disclosed connectors and systems are non-disposable.

In one or more embodiments, the herein disclosed connectors include a locking mechanism that can lock together the connector to the vessel. In one or more embodiments, the locking mechanism includes a locking member and a securing member. In one or more embodiments, the herein disclosed connectors and/or vessels comprise at least 2 locking mechanism and/or at least 2 engagement mechanisms.

In one or more embodiments, the locking member is configured to attach a securing member and the securing member configured to hold the locking member. Various forms of the locking members are herein contemplated. Non-limiting examples of the locking members include a hook, a tooth, a pin, a ratchet tooth, a knob, a ball, a thread, a bangle, a locking key, a stopper, and a zip-tie. The locking member is optionally selected from a structure that protrudes from a surface and grips a matching dent.

The locking member may be optionally moveable between a retracted position and an extended position. Such design allows to conveniently engage the vessels to each other, optionally or additionally allows to conveniently engage a connector to at least one vessel to each other, and while doing so, retracting the locking mechanism until positioning thereof to lock into the securing member. Optionally, the locking member extends from the securing member. For example, in cases where the securing member is a hole or the alike, the locking member may be positioned to extend from the hole. Various forms of the securing member are herein contemplated. Non-limiting examples of the securing member include a dent, a hole, a channel and a slot.

In one or more embodiments, the locking mechanism allows a secure (i.e., the connection is stable and does not spontaneously detach) and reversible locking (i.e., by a purposely initiated action of user). In accordance with this embodiment, the herein disclosed devices and systems may be reused, and the connection may be unlocked. In one or more embodiment, the locking mechanism may allow a permanent connection (i.e., the connection cannot be detached by a simple action and requires special instrumentation to detach). In one or more embodiments, the locking mechanism may optionally allow an irreversible connection (i.e., the resulting connection cannot be detached, unless the locking mechanism is destructed). In accordance with those embodiments, the herein disclosed devices and systems are intended for a single use.

Various types, combinations and numbers of locking members and securing members may be provided in each of the herein disclosed systems, to ensure the locking action. For example, a combination of two locking mechanisms or more may be provided in each connector/system/vessel.

The locking mechanism may be provided to lock or connect a connector to a vessel. In accordance with this embodiment, the locking member may be disposed on the connector and the securing member on the vessel, or vice versa. Various positions of the securing/locking member on the vessels or connectors are herein contemplated. The locking/securing member may be disposed on a body, port, and/or aperture, and/cavity of the connector and/or vessel. The locking mechanism may be disposed in an external or an internal position of the vessel and/or connector.

Optionally, the locking mechanism may be provided to lock or connect between a vessel to a connector or a connector to a cartridge. In accordance with this embodiment, the locking member may be disposed on the vessel/cartridge and the securing member on the connector, or vice versa. Various positions of the securing/locking member on the vessel/cartridge/connector are herein contemplated. The locking/securing member may be disposed on a vessel's/cartridge's/connector's body, port, aperture, and/or cavity. The locking mechanism may be disposed in an external or in an internal position of the vessel/cartridge/connector.

Optionally, the vessel-connector and/or connector-cartridge connection may include in addition to the locking mechanism an engagement mechanism configured or adapted to allow an engagement between the vessels and connectors. The engagement mechanism may be reversible, allowing disconnection of the vessel-connector. Various types of engagement mechanisms may be applicable and are contemplated. For example, the engagement mechanism is selected from, without limitation, a thread, a luer, a luer-lock, a luer-slip, snap-on, twist-on, a ratchet teeth mechanism, a retention member, a clamping rail, and an adhesive mechanism. The thread and the complementary thread may be selected from, without limitation, a luer, a smart-site mechanism and combinations thereof. In an embodiment, the engagement mechanism may be selected from a ratchet teeth mechanism, a clamping rail mechanism and/or an adhesive.

In one embodiment, the invention provides devices and systems configured to allow a substantially decontaminated locking of/engagement between vessels. In one or more embodiments, the devices and systems of the invention allow a substantially decontaminated fluid transfer between the one or more vessel(s) and or vessels and connectors. The engagement and/or locking and fluid transfer between the one or more vessels may accordingly be utilized via the connector which is substantially sterile and also allows the fluid transfer.

The connectors of the invention include a structure allowing connection to two vessels. The connectors include one or more walls, e.g., a side wall, a top wall and/or a bottom wall. The connectors configured to be coupled to two or more vessels and include applicable cavities for receiving the vessels. The cavities optionally formed by the side walls forming an inner lumen for accommodating therein the vessels. In one or more embodiments, the cavities of the connector may engulf the entire vessel's body. In one or more embodiments, the cavity engulfs a portion of a vessel. In one or more embodiments, the cavity of the connector engulfs at least a portion of a vessel's body. In one or more embodiments, the cavity of the connector engulfs at least a portion of a vessel's body and the port of the vessel. In one or more embodiments, the cavity of the connector engulfs a substantial portion of the vessel's body. In one or more embodiments, the cavity of the connector engulfs at least a substantial portion of the vessel's body and the port of the vessel. In one or more embodiments, the cavity of the connector engulfs the entire body of the vessel and the port of the vessel. In one or more embodiments, the cavity of the connector engulfs the entire length of the vessel. In one or more embodiments, the cavity of the connector engulfs the entire width of the vessel. In one or more embodiments, the cavity of the connector engulfs the entire length and width of the vessel. In one or more embodiments, the cavities engulf a port of a vessel or a portion thereof. The cavities optionally act further as a conduit via which medical substances may be transferred following engagement between, the vessels. Various sized and shapes of the connectors are applicable. For example, the connector may have a U-like structure. Alternatively, or additionally, the connector may have a shape of the vessel that should connect the connector. In one or more embodiments, the connector's cavity may have a shape that corresponds to the vessel's shape to allow the vessel to engage/couple with the connector's cavity. In one or more embodiments, at least a portion of the connector's cavity may have a shape that corresponds to at least a portion of the vessel's shape to allow the vessel to engage/couple with the connector's cavity. In one or more embodiments, the vessel is a vial. In one or more embodiments, the vessel is a container. In one or more embodiments, the vessel is a syringe. In one or more embodiments, the vessel is a cartridge. In one or more embodiments, the vessel is a bottle. In one or more embodiments, the vessel is a container with a protective side wall and a plunger. In one or more embodiments, the vessel having a plunger and/or a moveable wall.

In one or more embodiments, the connectors may be manufactured from various materials. Optionally, the connector is manufactured from an inert material, thus suitable for various types of pharmaceutical substances. The connector may be made from flexible or from rigid materials. Suitable materials include, without limitation a plastic, a glass, a rigid plastic, a flexible plastic, and combinations thereof. In one or more embodiments, the connector may be transparent. In one or more embodiments, the connector may be opaque. In one or more embodiments, at least a portion of the cavity of the connector may be made of a rubber material to provide an airtight/hermetic engagement between the connector and a vessel.

Various sizes and shapes of the connector are applicable. For example, the connector may have an external most length, width, and/or height of about 6 inches or less, 5 inches or less, 4 inches or less, 3 inches or less, 2 inches or less, or 1 inch or less.

The connector may include two or more cavities for accommodating two or more vessels. For example, the connector may include two, three, four, five, six, or more cavities.

In one or more embodiments, the connector is conveniently light presenting a weight of no more than about 100 grams, no more than about 50 grams, no more than about 25 grams, no more than about 15 grams, no more than about 10 grams, or no more than about 5 grams.

In one or more embodiments, the connector may be a closed structure which forms a tight, optionally, airtight connection with vessels. In one or more embodiments, the cavities of the connector are sealed by a cover/seal prior to an engagement or connection with a vessel. The seal or cover may be opened at the time of engagement, optionally by the engagement itself. Alternatively, or additionally, the seal or cover may be opened prior to any engagement or connection to a vessel. In one or more embodiments, when the cavities of the connector are connected to vessels or when covered by a cover or a seal, the interior of the connector is hermetically sealed/isolated from surrounding/environmental air. In one or more embodiments, the cover or the seal is frangible, breakable or rupturable. In one or more embodiments, the cover or the seal is pierceable by a piercing member such as a needle. In one or more embodiments, the cover or the seal is peelable.

In one or more embodiments, the system or connectors of the invention is provided when the connector is already connected to one or more vessels. In accordance with this embodiment, the one or more vessels may be integrally manufactured or assembled with the connector and provided or distributed to consumers as such. In one or more embodiments, at least one connector may form a unitary structure with a vessel. In accordance with these embodiments, the system/connector may be configured to allow a connection/locking/engagement to additional one or more vessels.

In one or more embodiments, the connector includes one cavity. In one or more embodiments, the connector includes two or more cavities disposed one above the other (i.e., parallel to each other) or which are longitudinally aligned with respect to each other (i.e., at opposing sides of the connector). In accordance with these embodiments, the vessels connected to such cavities are positioned aligned allowing a direct and aligned fluid passageway between the vessels. In one or more embodiments, the connector includes two or more ports which are shifted from each other. Yet, in accordance with this embodiment, a fluid passageway is formed between vessels which is twisted or curved. In yet one or more embodiments, the connector contains a plurality of cavities configured for connecting a plurality of vessels and/or providing a fluidic passageway between the plurality of the vessels.

In one or more embodiments, the herein disclosed invention allows transferring medical substances in a contaminant-free, or in a substantially contaminant-free manner.

In one or more embodiments, the herein disclosed invention affords an engagement of vessels in a contaminant-free, or in a substantially contaminant-free manner. In one or more embodiments, the herein disclosed invention affords an engagement of vessels in an air-free, or in a substantially air-free manner. In one or more embodiments, the herein disclosed invention affords locking of vessels in a contaminant-free, or in a substantially contaminant-free manner. In one or more embodiments, the herein disclosed invention affords locking of vessels in an air-free, or in a substantially air-free manner. In one or more embodiments, the herein disclosed invention provides a fluidic passageway or communication between vessels in a contaminant-free, or in a substantially contaminant-free manner. In one or more embodiments, the herein disclosed invention provides a fluidic passageway or communication between vessels in an air-free, or in a substantially air-free manner. In one or more embodiments, the herein disclosed invention, allows to substantially decrease the chances to introduce contaminants within a medical substance, when preparing or administering medical substances for administration to patients. As used herein the term “contaminant-free” is interchangeable with the term “sterile”, “disinfected”, and “decontaminated”. The term refers to substances that are free or substantially free of ambient air particles and/or pathogens and/or micro-organisms, and/or any of the alike. Typically, when less or no air is introduced within medical substances, the chances of contamination by pathogens, such as, bacteria, viruses, funguses, spores, pyrogens or the alike is completely abolished or significantly reduced.

As used herein, the term “substantially contaminant-free” means significantly less ambient air present when transferring medical substances with the herein disclosed vessels and systems, as compared to comparable conditions for transferring medical substances without the herein disclosed vessels and systems.

As used herein the term “ambient air particles” is interchangeable with the term “environmental air particles” and refers to air particles present in a non-filtered environment. The air particles may contain dirt, such as dust. Alternatively, or additionally, the air particles may contain pathogens or other microorganisms, or any of the alike.

As used herein the term “substances” refers to various types of materials. The substances may be liquid, semi-solid, or gas. In one or more embodiments, the substances are “medical substances”. As used herein the term “medical substances” refers to and encompasses any of the various pharmaceutical drugs, fluids, nutritional products and the like. In an embodiment of the invention the term “medical substance” is interchangeable with the term “pharmaceutical substance”.

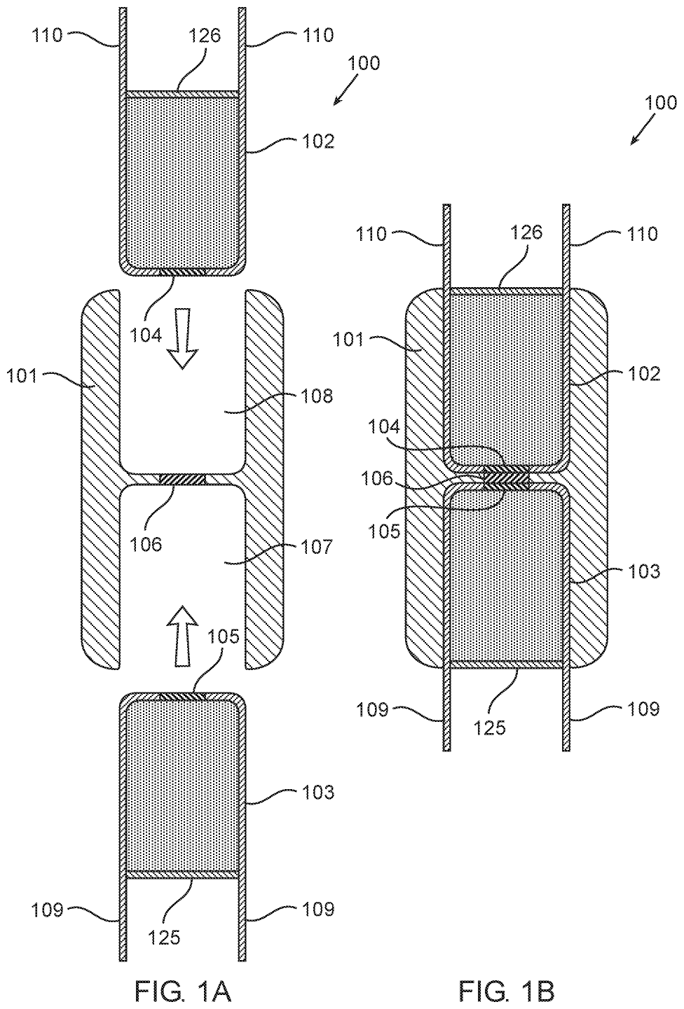

FIGS. 1A-1B illustrate a system 100 with a connector 101, a first vessel being cartridge 102 and a second vessel being cartridge 103. Although first and second vessels are cartridges, various alternative vessels are herein contemplated and applicable. Non-limiting examples of vessels include, a vial, a syringe, a bag, a bottle and the alike. The cartridges 102 and 103 configured to contain a fluid or semi fluid substance and to fill or refill each other. Optionally, or alternately, cartridges 102 and 103 may contain a solid substance such a powder, a lyophilized powder, a powdered medicament, etc. For example, first cartridge 102 may be provided empty and refilled or filled by second cartridge 103, or vice versa. In one or more embodiments, cartridges 102 and 103 (or vessels 102 and 103) may be prefilled by a drug/pharmaceutical manufacturer and/or distributer.

The connector 101 hence includes a first cavity 108 for accommodating or receiving a first cartridge 102 and a second cavity 107, for accommodating or receiving a second cartridge 103. The connector 101 is thus configured such to accommodate therein or engulf the bodies of at least two cartridges and/or vessels.

An aperture 106 is disposed between the cavities 107 and 108 for allowing passage of fluids (or solids/powdered solids) therethrough and between the cartridges. Cartridge 103 includes a cartridge aperture 105 and cartridge 102 includes a cartridge aperture 104. The connector aperture 106, first cartridge aperture 104 and/or second cartridge aperture 105 may include a seal and/or a cover which can be opened to allow fluid transfer between the cartridges 102 and 103. The seal and/or cover of any of aperture(s) 106, 104, and 105 may optionally be a frangible seal, a rupturable seal, a peelable seal, a pierceable seal, and combinations thereof. As illustrated in FIG. 1B, side walls 110 of cartridge 102 may be elongated and may extend outwardly from the side walls of connector 101. Similarly, side walls 109 of cartridge 103 may extend outwardly from the side walls of connector 101 when the cartridges are disposed within the connector 101. Inner walls 125 and 126 of cartridges 103 and 102, respectively may be moveable, optionally slidable to allow pushing the fluids out of the cartridges and establish the fluid transfer. Side wall 109 may in one or more embodiments additionally have an extra side wall (not shown) disposed along the inner portion of side wall 110. This extra side wall may be attached to inner wall 126 of cartridge 102 thus providing an extra (protective side wall that protects a user from contacting any substance contained in vessel/cartridge 102. Side wall 110 may be a circumferential side wall. The extra side wall (not shown) positioned parallel to side wall 110 on the interior portion of side wall 110 may also be a circumferential side wall. The same is applicable to cartridge 103 and its side wall 109.

An audible sound such as a “click” sound or any other sound may be emitted at about the time, or at the time of connection of cartridge 102 and/or 103 with connector 101.

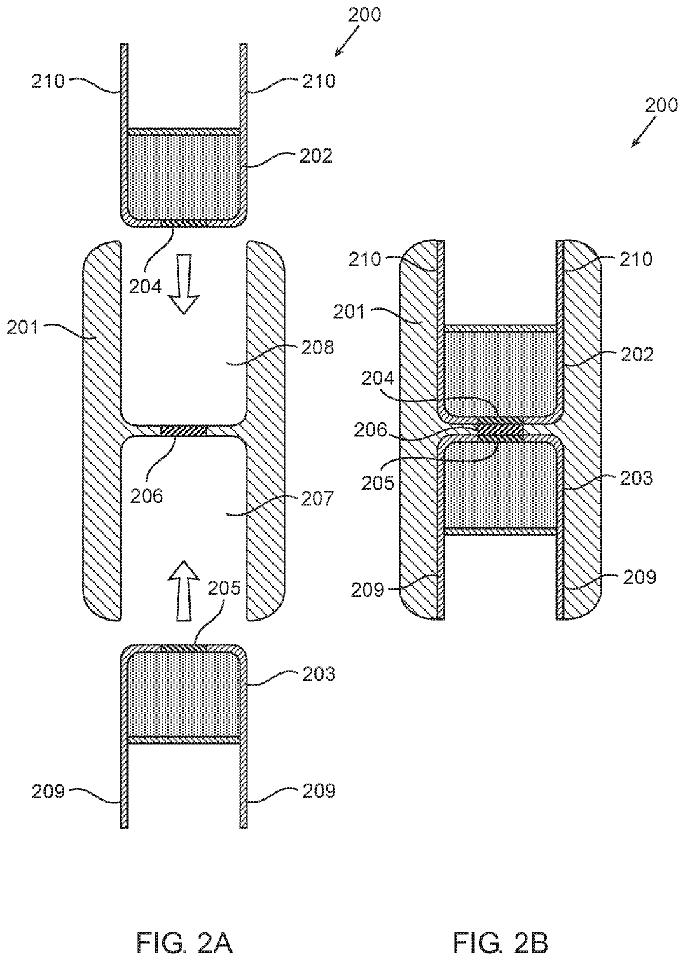

FIGS. 2A-2B, illustrate a system 200 which is similar to system 100 but includes cartridges 202 and 203 with side walls 210 and 209, respectively, that are fully engulfed within the side walls of the connector 201 and when loaded into the connector 201 are aligned with the walls of the connector 201. That is, cavities 208 and 207 are sized and shaped to fully accommodate (engulf) the bodies of cartridges 202 and 203. That is, cavities 208 and 207 are sized and shaped to fully accommodate (engulf) the bodies and side walls 210 and 209 of cartridges 202 and 203.

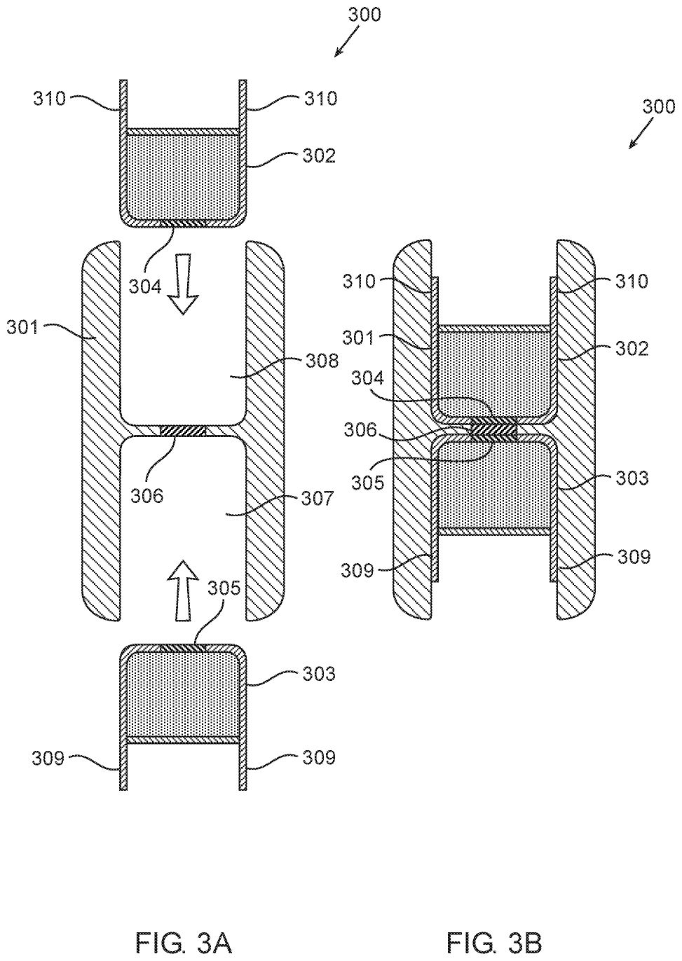

FIGS. 3A-3B illustrate system 300 which is similar to systems 100 and 200, except for side walls 309 and 310 of cartridges 303 and 302, respectively, which are shortened and concealed within the side walls of the connector 301 when the cartridges 302 and 303 are loaded into the connector 301.

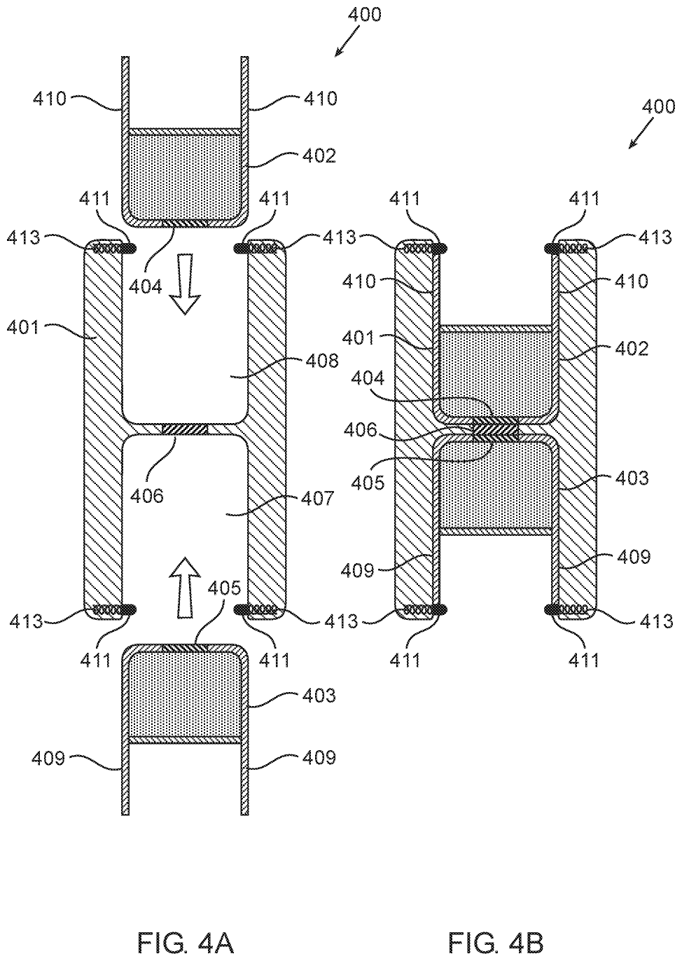

FIGS. 4A-4B illustrate system 400 which includes a connector 401 and cartridges 402 and 403. The system 400 further includes a locking mechanism in the form of stoppers 411 attached to springs 413 which secure the connection of the cartridges 402 and 403 to connector 401. The stoppers 411 are retractable upon the engagement or insertion of the cartridges within the connector 401 and extendible to lock the cartridge to the connector, after the insertion of the cartridges within the connector 401. Once attached, a fluid communication can be obtained between the cartridges 403 and 402 via apertures 404, 406 and 405.

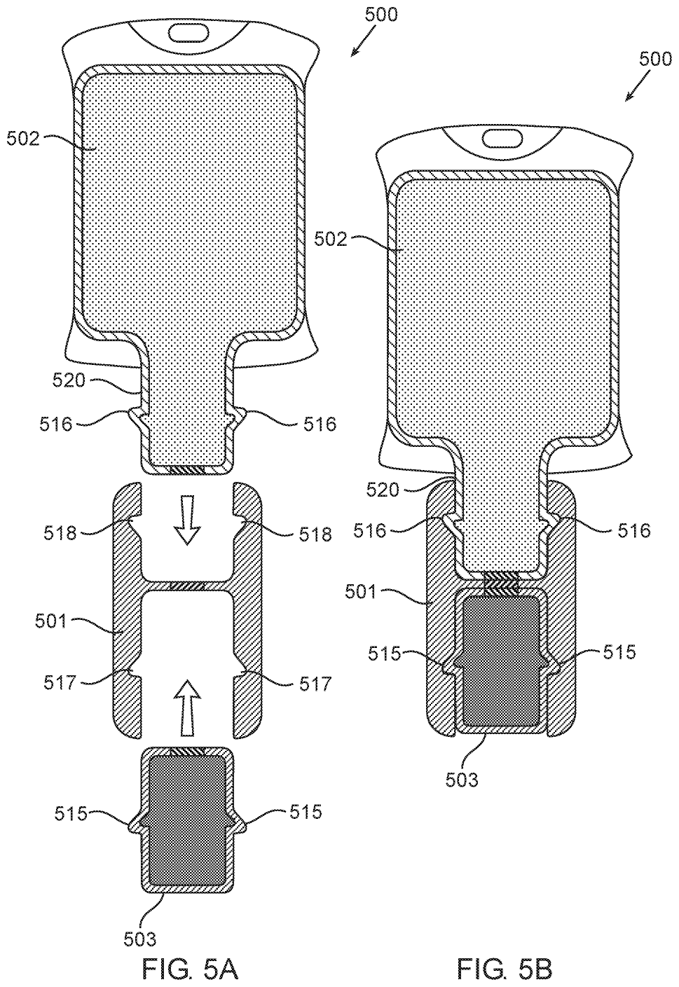

FIGS. 5A-5B illustrate yet another system 500 with connector 501 and two vessels, being bag 502 and cartridge 503. Here, the connector 501 is configured to accommodate the entire body (entire length of body) of cartridge 503 and to further connect port 520 of bag 502. A locking mechanism in the form of teeth 515 disposed on opposing side wall of cartridge 503 is provided which can be locked into dents 517 disposed within the (bottom) cavity of connector 501. Similarly, a locking mechanism in the form of teeth 516 is provided disposed on port 520 of bag 502, the teeth 516 which can engage with dents 518 disposed within (top) cavity of connector 501. Once attached, a fluid communication can be obtained/established between the cartridge 503 and the bag 502. Bag 502 is used arbitrarily and may be any vessel such as a vial, a bottle, a syringe, etc. Similarly, cartridge 503 is used arbitrarily and may be a vessel such as a vial, a bottle, a syringe, etc.

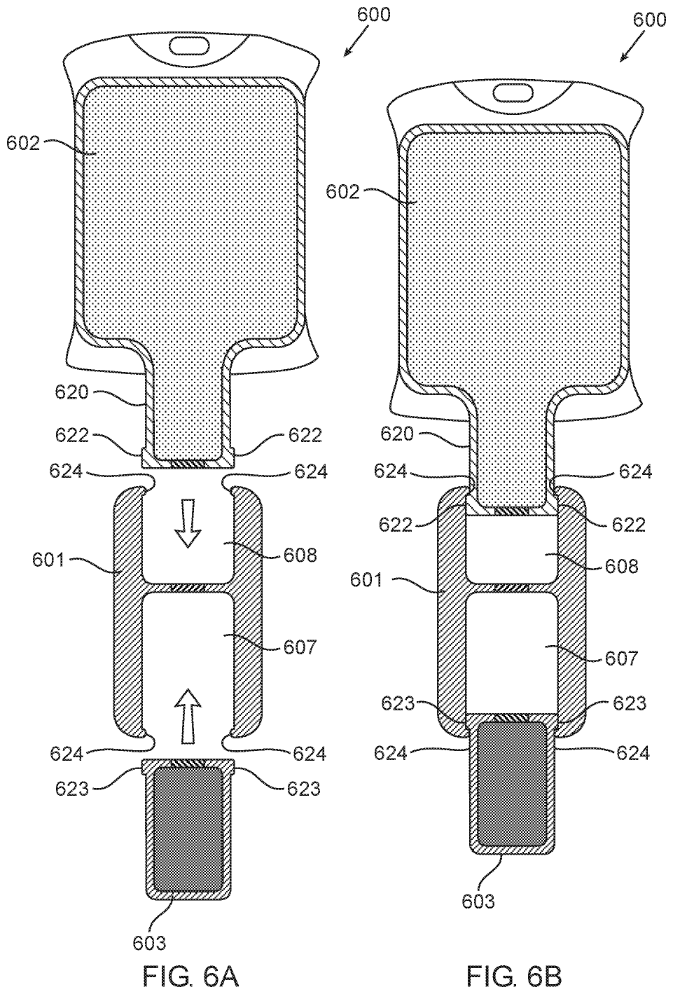

FIGS. 6A-6B illustrate yet another connector 601 which is similar to connector 501 but includes an alternative engagement mechanism. Here, the connector 601 includes side tabs 624 protruding into cavities 608 and 607 at a bottom wall and upper wall of the connector 601. Cartridge 603 includes corresponding extensions 623 which protrude outwardly from a top end of side walls of the cartridge 603. Optionally, or alternately, cartridge 603 may include extensions 623 protruding outwardly (not shown) from a bottom end of side walls of cartridge 603. Similarly, bag 602 includes corresponding extensions 622 which protrude outwardly from an outer surface of side walls of port 620. The extensions 622 and 623 can lock into connector 601 by a friction match when connecting tabs 624.

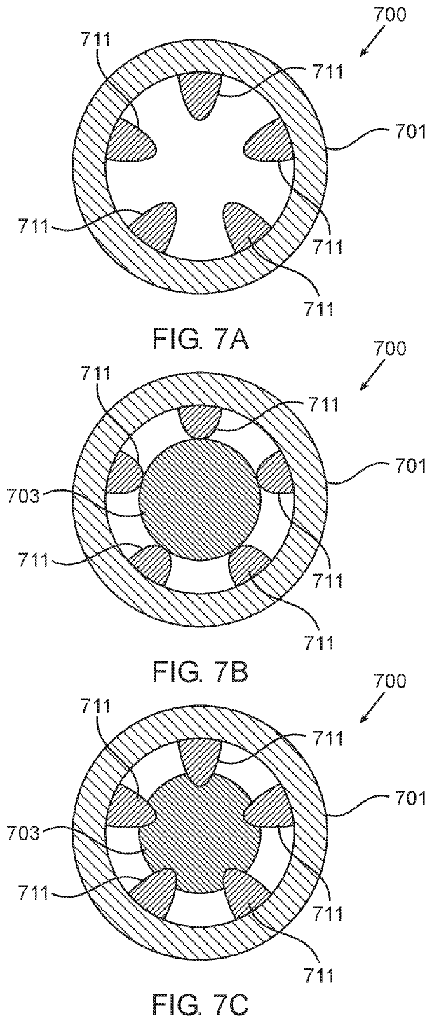

In FIGS. 7A-7C an engagement mechanism in the form of retractable stoppers 711 is shown, from a bottom view of connector 701. Retractable stoppers 711 are shown in various stages. In FIG. 7A, the stoppers 711 are shown when in an extended position prior to any insertion of a cartridge 703 and in FIG. 7C, the stoppers 711 shown after the cartridge 703 has been positioned entirely within the connector 701. Upon the engagement of the cartridge 703 into connector 701, the stoppers 711 retract, allowing the insertion of the cartridge 703 within the connector 701 (FIG. 7B). The stoppers 711 extend back to their initial position after the loading of the cartridge 703 within the connecter 701 as shown in FIG. 7C. In one or more embodiments, stoppers 711 may be made of a plastic material, a rubber material, a metal material, and combinations thereof. In one or more embodiments, stoppers 711 may be ratchet teeth. In one or more embodiments, the stoppers 711 may be made of a rigid plastic material that is configured to be slightly flexible to allow entry of the cartridge 703 into connector 701 but rigid enough to hold cartridge 703 within connector 701.

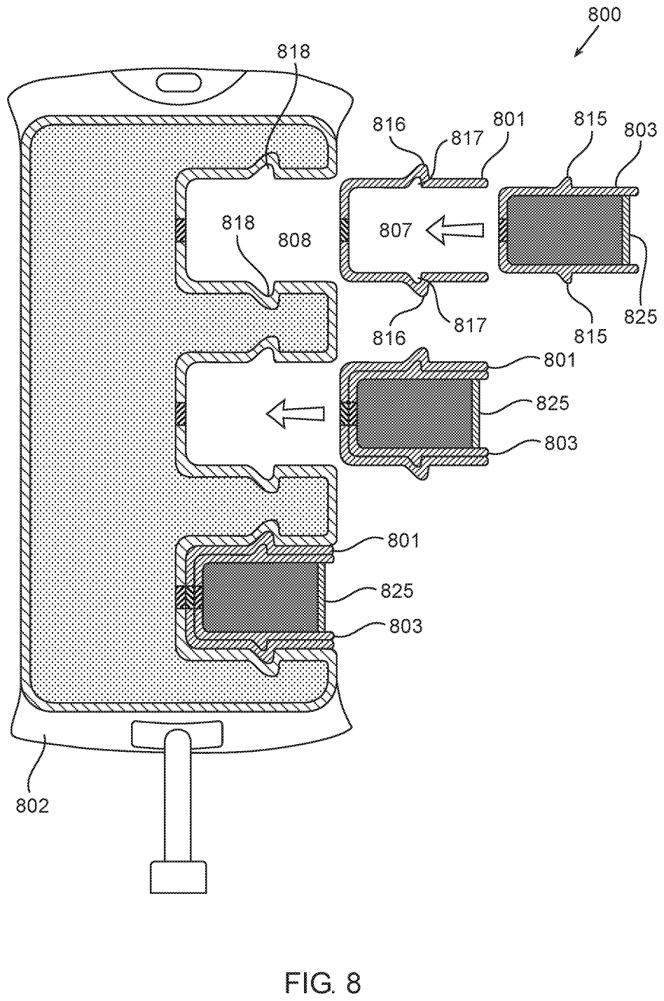

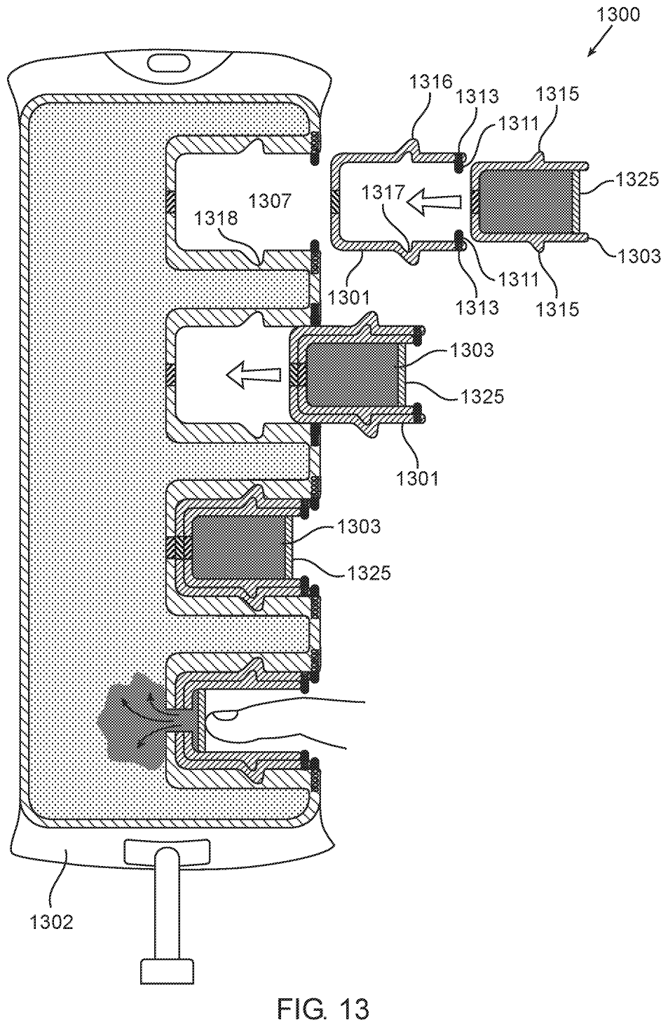

FIG. 8 illustrates yet another exemplary system 800 which includes a bag 802 with three cavities 808 for accommodating or receiving (engulfing) therein three cartridges 803 via connectors 801. Three connectors 801 are provided in order to allow or mediate the connection between the cartridges 803 and vessel 802. Although, the system 800 is illustrated to contain three cartridges 803 and one vessel 802, alternative number of cartridges and/or vessels are contemplated. Applicable configurations may include two, four, five, six or more cartridges that can be connected to a vessel via connectors. A locking mechanism in the form of teeth 815 is provided to allow a secure connection between the cartridges 803 and the connectors 801. Teeth 815 protrude from the external surface of the side walls of cartridges 803 and can engage into their respective connectors 801 via dents 817. Optionally, the shape of dents 817 forms external teeth 816 which protrude from the side walls of the connectors 801 and those teeth 816 can connect and lock into dents 818 within cavities 808 of vessel 802. Cavities 808 are sized and shaped such to entirely engulf and conceal the bodies of cartridges 803 and connectors 801 upon the engagement of same within the vessel 802. The system 800 further includes a fluid transfer mechanism for effecting the fluid transfer between the vessels. A moveable wall 825, provided at the bottom of vessels 803 allows for the fluid transfer by manually pushing the wall 825 against an opposing wall to thereby forcibly push the substance out of the cartridge/vessel 803 and into vessel 802 (see also FIG. 13). Bag 802 in this figure may in one or more embodiments be any type of container such as a bottle. Optionally, bag 802 may be a manifold having a plurality of cavities 808, the cavities 808 configured to accommodate and/or receive (engulf) their respective connectors 801 and cartridges 803.

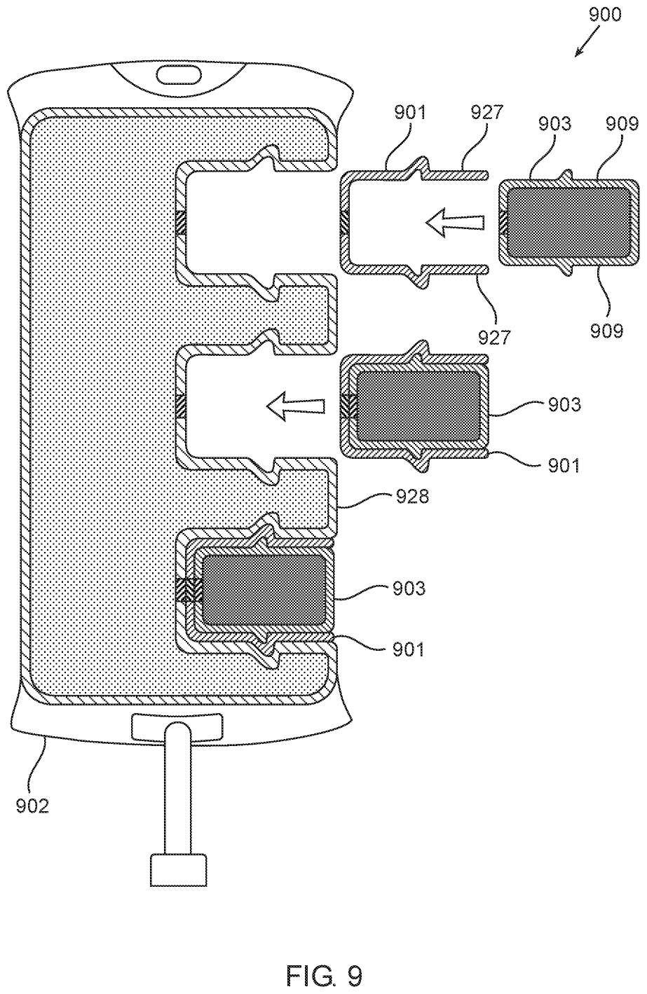

FIG. 9 illustrates a system 900 which is similar to system 800 but here the side walls 909 of the cartridges 903 as well as the side walls 927 of connectors 901, align with the side wall 928 of the vessel 902, upon the engagement between the connectors 901 and cartridges 903 with vessel 902. Vessel 902 is shown as a bag but may be a bottle, any type of container, and optionally a manifold.

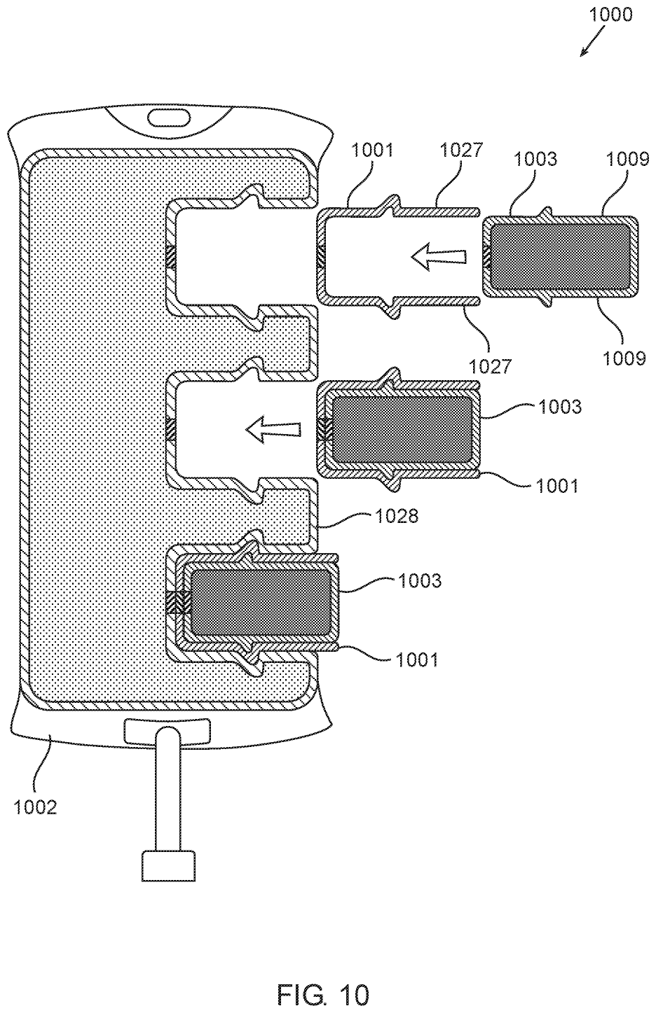

FIG. 10 illustrates system 1000 which is similar to systems 800 and 900, but here the side walls 1009 of the cartridges 1003 as well as the side walls 1027 of connectors 1001 are elongated and extend outwardly from the side wall 1028 of the vessel 1002 after the engagement between the vessel 1002 and the connectors 1001 and cartridges 1003. This figure illustrates how a substantial length of side walls 1009 of cartridges 1003 are disposed inside connector 1001 thus protecting a substantial portion of the body of cartridges 1003 from breakage if cartridges 1003 happen to fall to the ground. Thus connector 1001 having a cavity that accommodates (engulfs) a substantial length and/or volume of cartridges 1003 is important for protecting breakage of cartridges 1003 during the preparation and/or administration of beneficial and/or medical substances. Vessel 1002 is shown as a bag but may be any type of container such as a bottle, etc. Vessel 1002, in one or more embodiments, may be a manifold. Cartridges 1003 may be any type of containers and/or medical devices such as vials, syringes, bottles, containers with plunger, containers with protective side walls, etc.

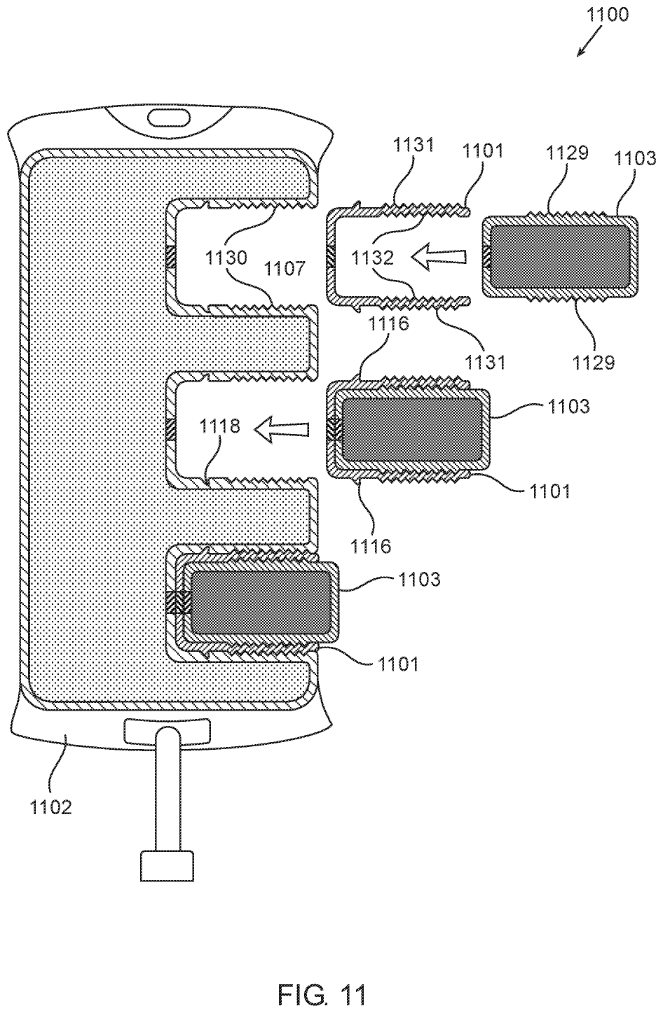

FIG. 11 illustrates yet another system 1100 which includes an engagement/locking mechanism in the form of threads which can engage with cooperating threads. For example, cartridge 1103 includes side threads 1129 at/on the outer surface of the cartridge's body. The side threads 1129 may cover a portion of the surface of the cartridge's body. The side threads 1129 in some embodiments may be circumferential threads surrounding at least a portion and/or the entire surface of the cartridge's body. The threads 1129 can engage (are configured to engage) with connector 1101 via internal threads 1132 within a cavity of the connector 1101. Connector 1101 further includes external threads 1131 which can engage with vessel 1102 via internal threads 1130 disposed in cavity 1107 of vessel 1102. The system 1100 further includes an additional locking mechanism in the form of teeth 1116 disposed on external portion of connector 1101 configured to engage matching dents 1118 disposed in/on cavity 1107 to further secure the connection between the connector 1101 and the vessel 1102.

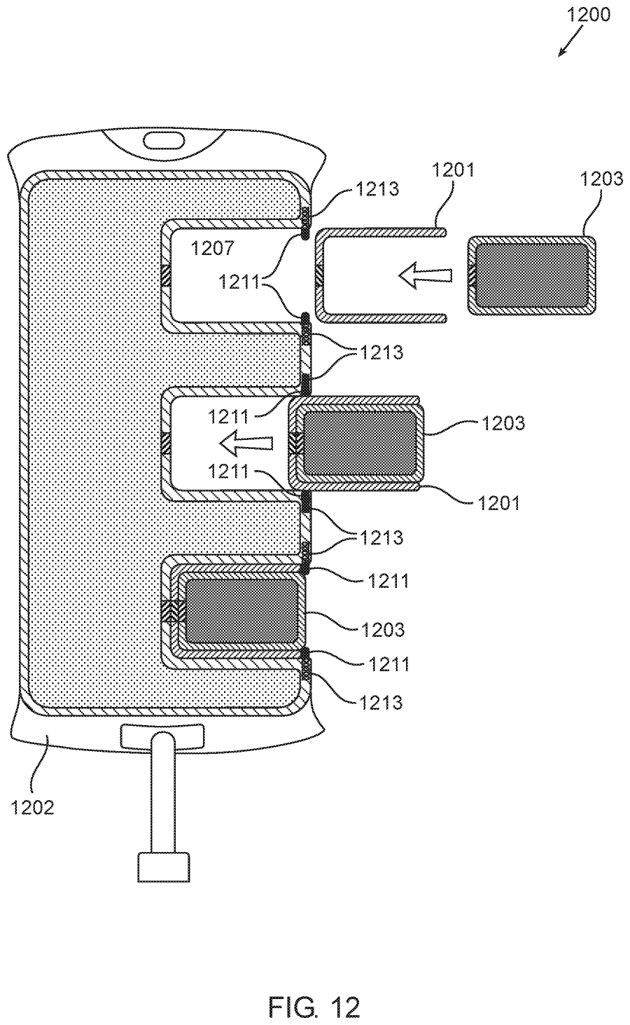

FIG. 12 illustrates system 1200 with an engagement mechanism comprising retractable stoppers 1211 that are configured to lock or allow the engagement between vessel 1202, connectors 1201, and optionally also vessels 1203. Stoppers 1211 extend from opposing side ends of cavity 1207 of the vessel 1202 and are retractable, optionally into a dedicated channel within the side wall where springs 1213 reside. The insertion of the connectors 1201 into the cavities 1207 causes retraction of the stoppers 1211 into their dedicated channels via the springs 1213 and the cartridges 1203 and connectors 1201 may be introduced (may enter) into cavities 1207. Once fully inside the cavities 1207, the springs 1213 expand and push stoppers 1211 outwardly to their initial position to thereby lock the cartridges 1203 and the connectors 1201 within the vessel 1202.

FIG. 13 illustrates system 1300 which includes four cavities 1307 for accommodating four cartridges 1303 via connectors 1301. The system includes the following two locking mechanisms for locking cartridge 1303 to connector 1301: (i) side teeth 1315 which can be locked into dents 1317; (ii) retractable stoppers 1311. The teeth 1315 disposed on the outer surface of the side walls of cartridge 1303 and those teeth may engage with connector 1301 via dents 1317. Retractable stoppers 1311 which can lock cartridge 1303 within the connector's cavity are disposed on opposing internal ends of connector 1301. A locking mechanism of teeth 1316 and dents 1318 is provided to secure the connection between the connector 1301 and the vessel 1302.

The fluid transfer mechanism of the system 1300 includes moveable wall (plunger) 1325 which when pushed by a user allows the fluid transfer between the cartridges 1303 and the vessel 1302. Optionally, wall 1325 is moveable via a sliding mechanism including rails disposed on/in cartridge 1303.

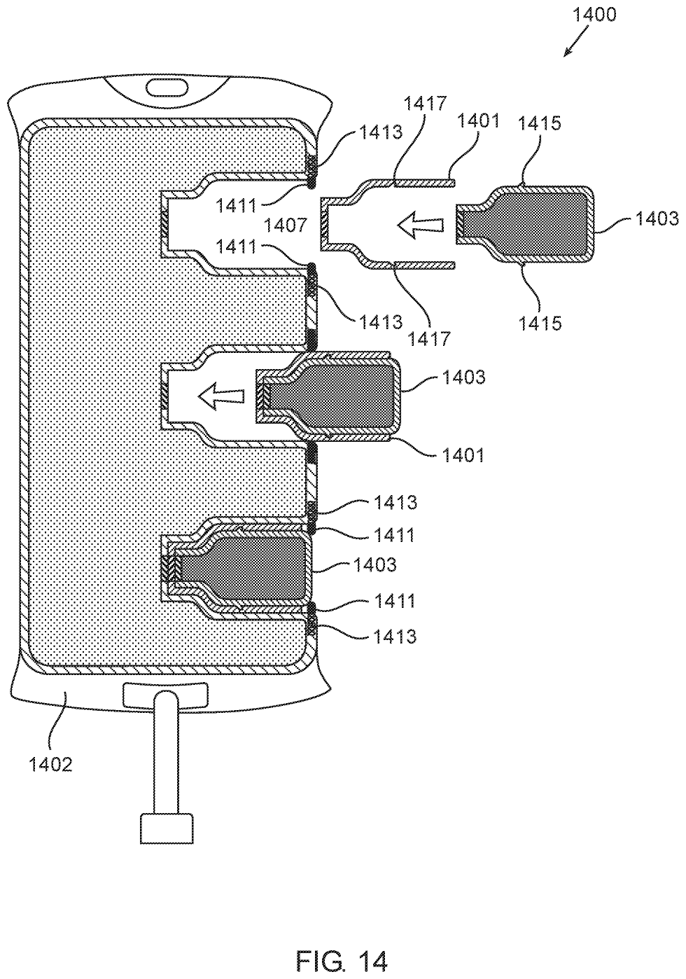

FIG. 14 illustrates system 1400 with connectors 1401 having a structure/shape and/or size of a vial and which can accommodate cartridge 1403 having a structure of a bottle/vial. A locking mechanism including teeth 1415 and dents 1417 allows the secure engagement between the cartridges 1403 and connectors 1401. The system further includes a locking mechanism of spring 1413 loaded retractable stoppers 1411, for securing the engagement of the connectors 1401 with bag 1402.

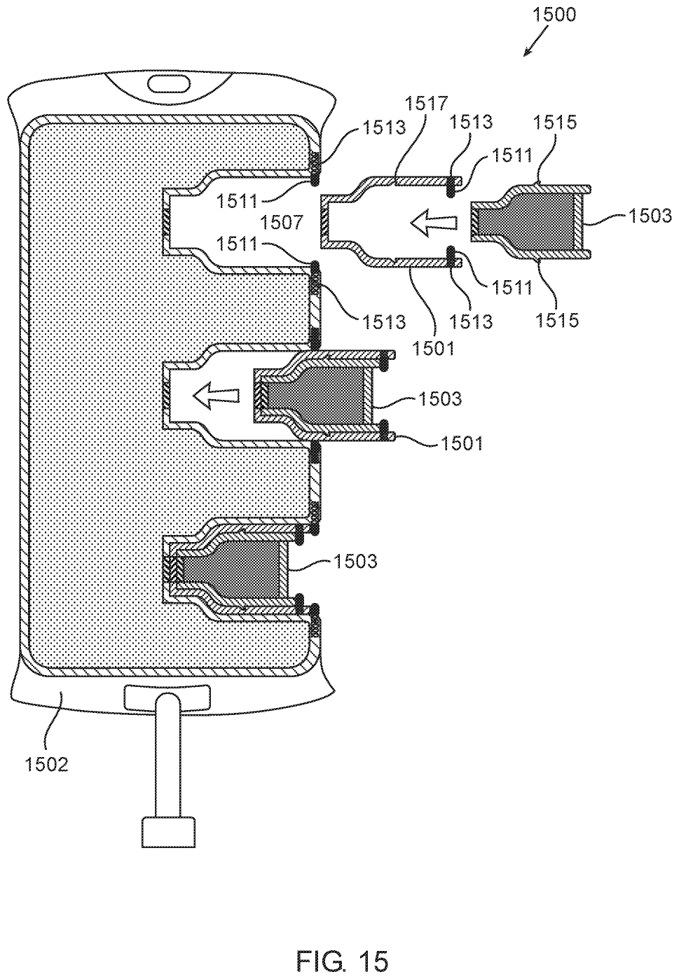

FIG. 15 illustrates a similar system 1500 which includes two locking mechanisms for securing the engagement between the connector 1501 and cartridge 1503 is shown in FIG. 15. The system 1500 includes teeth 1515 on the side walls of cartridge 1503 which can lock into dents 1517 and stoppers 1511 on connector 1501. Similar stoppers 1511 are provided on the side wall of bag 1502 in order to secure the connection between the bag 1502 and the connector 1501. Connector 1501 is shown having a cavity configured to accommodate the entire length, width, and/or volume of cartridge (vessel) 1503 thus protecting cartridge (vessel) 1503 from breakage/cracking during the assembly/preparation/administration of a beneficial substance.

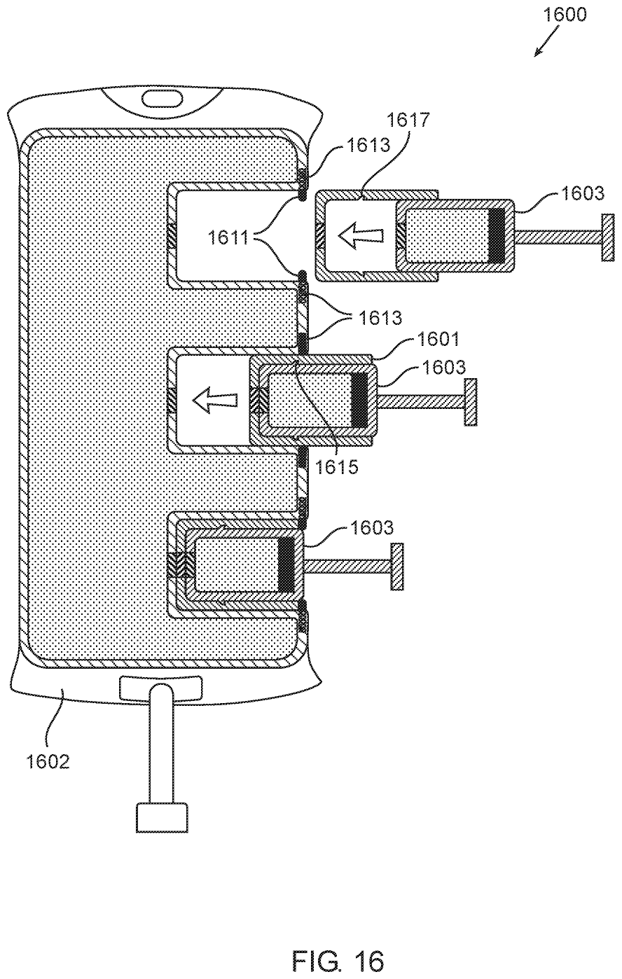

FIG. 16 illustrates system 1600 which shows yet another exemplary connector 1601 and cartridge 1603 which in this case has a form of a syringe. The syringe 1603 engages vessel 1602 via connector 1601. A locking mechanism of teeth 1615 disposed on a surface of syringe 1603 is configured to engage dents 1617 disposed within a cavity of connector 1601 to provide a safe and secure connection between syringe 1603 and connector 1601. Stoppers 1611 provide safe connection between the connector 1601 and syringe 1603 to vessel 1602.

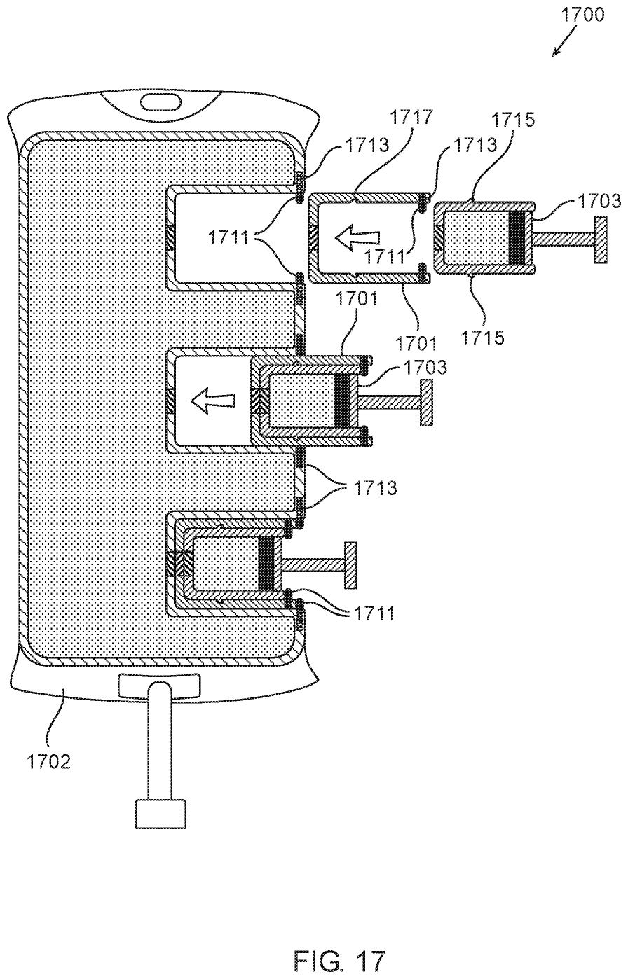

FIG. 17 illustrates system 1700 in which the system may include yet two locking mechanisms which can include teeth 1715-dents 1717 and stoppers 1711 for the engagement between the syringe 1703 and connector 1701, and additionally engagement of connector 1701 to vessel 1702.

Each of the following terms: ‘includes’, ‘including’, ‘has’, ‘having’, ‘comprises’, and ‘comprising’, and, their linguistic, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably.

The term ‘about’, is some embodiments, refers to ±30% of the stated numerical value. In further embodiments, the term refers to ±20% of the stated numerical value. In yet further embodiments, the term refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A system for the engagement and fluid passageway between vessels, the system comprising:
a first vessel;
a second vessel; and
a connector having at least one cavity, the cavity configured to allow entry of at least one of the first vessel's body and the second vessel's body, the connector configured to allow a connection between the first vessel and the second vessel;
wherein the first and second vessels connected to each other via the connector and wherein each of the first vessel, second vessel and connector includes a sealed aperture for allowing the fluid passageway between the first vessel and the second vessel.

2. The system of claim 1, wherein the connector includes at least one cavity, the cavity sized and shaped to accommodate at least one of a portion of the first vessel's body and the second vessel's body.

3. The system of claim 2, wherein the connector includes a first cavity for accommodating therein at least 25% of a length of the first vessel and a second cavity for accommodating at least 25% of a length of the second vessel.

4. The system of claim 1, wherein the first and/or second vessels include a cavity for accommodating therein the connector or a portion thereof, thereby allowing the vessels connection and fluid passageway via the connector.

5. The system of claim 2, wherein the connector includes a cavity for accommodating therein the first vessel and wherein the second vessel includes a cavity for accommodating therein the connector, thereby allowing the vessels' connection and fluid passageway via the connector.

6. The system of claim 1, which comprises a plurality of first vessels and a plurality of connectors, such that each first vessel configured to connect a connector and the second vessel configured to connect the plurality of connectors and vessels.

7. The system of claim 6, wherein the second vessel includes at least two cavities for accommodating therein at least two of the connectors.

8. The system of claim 7, wherein at least two of the connectors includes a cavity for accommodating therein at least two of the first vessels.

9. The system of claim 1, wherein the vessel is selected from the group consisting of a bottle, a syringe, an infusion line, a connector, a filter, a manifold, a bag port, a bottle port, a vial port, a cartridge and a combination thereof.

10. The system of claim 9, wherein the first vessel is a cartridge configured to fill the second vessel with a fluidic or semi fluidic substance.

11. The system of claim 1, wherein when connected, a wall of at least one of the first vessel and second vessel extends from the boundaries of a wall of the connector.

12. The system of claim 1, wherein when connected, a wall of at least one of the first vessel and second vessel is aligned with the boundaries of a wall of the connector.

13. The system of claim 1, wherein when connected, the walls of at least one of the first vessel and second vessel are concealed within the walls of the connector.

14. The system of claim 1, wherein when connected, a wall of the connector and/or the first vessel extends from the boundaries of a wall of the second vessel.

15. The system of claim 1, wherein when connected, a wall the connector and/or the first vessel is aligned with the boundaries of a wall of the second vessel.

16. The system of claim 1, wherein when connected, the walls of the connector and/or the first vessel are concealed within the walls of the second vessel.

17. The system of claim 1, wherein the first vessel and/or second vessel includes a fluid transfer mechanism for transferring a fluidic or semi fluidic substance from the first vessel to the second vessel or vice versa.

18. The system of claim 17, wherein the fluid transfer mechanism includes a moveable wall which when pushed towards an aperture of the vessel, forcibly pushes the fluidic or semi fluidic substance from the vessel.

19. The system of claim 1, further comprising a locking mechanism configured to secure the engagement between the vessel and connector.

20. The system of claim 19, wherein the locking mechanism comprises a securing member which is a tooth.

21. The system of claim 20, wherein the securing member is locked into a locking member selected from a dent.

22. The system of claim 20, wherein the locking member is retractable and moveable between a retracted position wherein the locking member abuts and/or inserted within a wall of the vessel and an extended position, wherein the locking member protrudes from a wall of the vessel.

23. The system of claim 20, wherein the locking member is retractable and moveable between a retracted position wherein the locking member abuts and/or inserted within a wall of the connector and an extended position, wherein the locking member protrudes from a wall of the connector.

24. The system of claim 1, wherein the locking member is spring loaded or attached to a spring, allowing planar movement of the locking member.

25. A connector for the engagement and establishment of a fluid passageway between vessels, the connector configured to connect a first vessel and a second vessel, the connector comprising at least one cavity for accommodating therein at least one of the first and second vessels.

26. The connector of claim 25, wherein the cavity is configured to allow insertion of a vessel's body into the cavity.

27. The connector of claim 26, the connector further comprising a dent disposed on a surface of the cavity, the dent configured to engage a tooth of a vessel.

28. The connector of claim 26, the connector further comprising at least one tooth configured to engage a dent disposed on a surface of a vessel's cavity.
